(19) United States
(12) Patent Application Publication
(10) Pub. No.: US 2006/0004211 A1
(43) Pub. Date: Jan. 5, 2006

(54) Preparation of 4-substituted-2-buten-4-olides from mucohalic acids

(75) Inventors: Paul Angell, Dexter, MI (US); Daniel Thomas Belmont, Howell, MI (US); Timothy Curran, Whitmore Lake, MI (US); Ji Zhang, Ann Arbor, MI (US)

Correspondence Address: WARNER-LAMBERT COMPANY, 2800 PLYMOUTH RD, ANN ARBOR, MI, 48105, US

(73) Assignee: WARNER-LAMBERT COMPANY LLC

(21) Appl. No.: 11/168,795

(22) Filed: Jun. 28, 2005

Related U.S. Application Data
(60) Provisional application No. 60/585,127, filed on Jul. 1, 2004.

(51) Int. Cl. C07D 307/34

(52) U.S. Cl. 549/313

(57) ABSTRACT

Methods and materials for preparing 4-substituted-2-buten-4-olides are disclosed. The method includes reacting a mucohalic acid with a silyl enol ether or a ketene silyl acetal in the presence of a Lewis acid.

### CROSS-REFERENCE TO RELATED APPLICATIONS

This application claims priority from U.S. Provisional Patent Application No. 60/585,127 filed Jul. 1, 2004.

### BACKGROUND OF THE INVENTION

### FIELD OF INVENTION

This invention relates to materials and methods for preparing 4-substituted-2,3-dihalo-2-buten-4-olides (γ-substituted γ-butenolides), which are useful intermediates for preparing biologically active natural products and compounds.

### DISCUSSION

Substituted γ-butyrolactones (γ-butanolides or γ-lactones) and α,β-unsaturated γ-lactones (γ-butenolides) have attracted much attention from medicinal and synthetic organic chemists. For discussions concerning the preparation of γ-butyrolactones, see P. V. Ramachandran et al., J. Org. Chem. 67:5315 (2002), B. W. Greatrex et al., J. Org. Chem. 67:5307 (2002), M. Movassaghi & E. N. Jacobsen, J. Am. Chem. Soc. 124:2456 (2002), J. Cossy et al., J. Org. Chem. 66:7195 (2001), M.-H Xu et al., J. Org. Chem. 66:3953 (2001), P. V. Ramachandran et al., Org. Lett. 3:17 (2001), D. Díaz & V. S. Martin, Org. Lett. 2:335 (2000), P. V. Ramachandran et al., Tetrahedron: Asymm. 10:11 (1999), B. M. Trost & Y. H. Rhee, J. Am. Chem. Soc. 121:11680 (1999), E. O. Martins & J. L. Gleason, Org. Lett. 1:1643 (1999), H. J. Ha et al., J. Org. Chem. 63:8062 (1998), A. M. Fernandez et al, J. Org. Chem. 62:4007 (1997), S. Fukuzawa et al, J. Am. Chem. Soc. 119:1482 (1997), and A. Vaupel & P. Knochel, J. Org. Chem. 61:5743 (1996). For discussions regarding the preparation of γ-butenolides, see S. P. Brown et al., J. Am. Chem. Soc. 125:1192 (2003), K. Suzuki & K. Inomata, Tetrahedron Lett. 44:745 (2003), S. Ma et al., Org. Lett. 2:1419 (2000), S. Ma & S. Wu, J. Org. Chem. 64:9314 (1999), and Y. Nagao et al., Ibid. 54:5211 (1989).

Gamma-butyrolactones and γ-butenolides appear in a variety of biologically active natural products and pharmaceuticals. See, e.g., C. Böhm & O. Reiser, Org. Lett. 3:1315 (2001) and M. Pohmakotr et al., Helv. Chim. Acta 85:3792 (2002) ((−)-roccellaric acid); A. Brecht-Forster et al., Helv. Chim. Acta 85:3965 (2002), T. P. Loh & P. L. Lye, Tetrahedron Lett. 42:3511 (2001), and S. Drioli et al., J. Org. Chem. 63:2385 (1998) (phaseolinic acid); H. A. Avedissian et al., J. Org. Chem. 65:6035(2000) (asimicin and bullatacin); S. C. Sinha et al., J. Org. Chem. 64:7067 (1999) (squamotacin); A. Sinha et al., J. Org. Chem. 64:2381 (1999) (trilobin); J. Zhang et al., Org. Lett. 4:4559 (2002) (rofecoxib); W. C. Patt et al., J. Med. Chem. 42:2162 (1999) and W. C. Patt et al., J. Med. Chem. 40:1063 (1997) (endothelin antagonists); and S. K. Bagal et al., Org. Lett. 5:3049 (2003) and S. K. Bagal et al., Tetrahedron Lett. 44:4993 (2003) (biatractylolide, biepiasterolide). See also J. D. McCombs et al., Tetrahedron, 44:1489 (1988).

In addition, γ-butyrolactones and γ-butenolides are prominent moieties in natural flavors and odors, including sex attractant pheromones of some species of insects, and may prove beneficial for developing environmentally friendly insecticides. See P. de March et al., J. Org. Lett. 2:163 (2000) and C. Harcken & R. Bruckner, Angew. Chem., Int. Ed. 36:2750 (1997). γ-butenolides have been employed to make some functionalized open-chain molecules, such as 1,4-solfanylalcohols, which are found in fruits and vegetables and have been the subject of intense research in flavor chemistry. See J. J. Filippi et al., Tetrahedron Lett. 43:6267 (2002). Furthermore, γ-butyrolactones also serve as precursors to fused bicyclic lactones. See M. J. Chen et al., J. Org. Chem. 64:8311 (1999) (dihydrocanadensolide, isoavenociolide, ethisolide), and S. Tsuboi et al., J. Org. Chem. 63:1102 (1998) (avenaciolide).

Mucohalic acids are highly functionalized molecules, which are thought to exist primarily in cyclic form (Formula 1), but may also exist in an open form (Formula 1′), 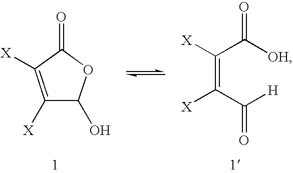
in which X is halogen (F, Cl, Br, or I). As such, they may be viewed as α,β-unsaturated aldehydes and pseudo unsaturated γ-lactones, which make them ideal building blocks for accessing highly functionalized γ-substituted γ-butenolides (i.e., 4-substituted-2-buten-4-olides). See J. Zhang et al., Tetrahedron Lett. 44:5579 (2003).

However, nucleophilic addition to the aldehyde carbonyl of mucohalic acid (see Formula 1′) to form γ-substituted γ-butenolides is difficult because vinyl halides are sensitive to nucleophiles. E. Beska & P. Rapos, J. Chem. Soc., Perkin Trans. 1 23:2470 (1976). Additionally, application of the classic aldol reaction, the base catalyzed condensation of one carbonyl-containing compound with the enolate/enol of another, is hampered by the observation that mucohalic acids have poor stability under basic conditions. These stability issues surrounding mucohalic acids suggest that a different approach is needed for preparing γ-substituted γ-butenolides from mucohalic acids.

One potentially useful approach relates to the so-called Mukaiyama aldol reaction, which is a Lewis acid catalyzed condensation of a carbonyl-containing compound with an enol equivalent. See C. H. Heathcock, Comp. Org. Syn. 2:133 (1991) and T. Mukaiyama, Org. React. 28:203 (1982). There appear, however, to be few reports regarding the use of this approach to form γ-butenolides. Feringa and coworkers reported the asymmetric synthesis of γ-substituted γ-butenolides via Mukaiyama aldol type reaction where (R)-5-(menthyloxy)-2(5H)-furanone was the chiral synthon. A. van Oeveren & B. L. Feringa, J. Org. Chem. 61:2920 (1996). Evans and co-workers describe the synthesis of enantiomerically pure γ-substituted γ-butenolides using a 2-siloxyfuran and C₂-symmetric Cu(II) complexes. D. A. Evans et al. J. Am. Chem. Soc. 121:669 (1991). R. Brückner and co-workers reported a Mukaiyama aldol addition/anti-elimination route to γ-alkylidenebutenolides using a 2-siloxyfuran as the starting material. F. von der Ohe & R. Brückner New J. Chem. 24:659(2000).

### SUMMARY OF THE INVENTION

The present invention provides methods and materials for preparing 4-substiuted-2,3-dihalo-2-buten-4-olides (γ-substituted γ-butenolides). The method is based on the Mukaiyama aldol reaction and involves the Lewis acid catalyzed condensation of a mucohalic acid (Formula 1) and a silyl enol ether (Formula 3, below). The claimed method may employ a chiral Lewis acid, which results in enantiomerically enriched products. The claimed method employs inexpensive, readily available starting materials (Formula 1) and permits easy access to γ-substituted γ-butenolides having α- and β-activated functional groups. The method should allow chemists to prepare complex molecules containing the γ-butenolide moiety.

One aspect of the present invention provides compounds of Formula 2, 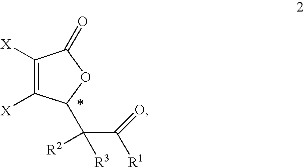
in which

X is halogen;

R¹ is C₁₋₆ alkyl, C₃₋₈ cycloalkyl, C₁₋₆ alkoxy, C₁₋₆ alkylthio, aryl, aryl-C₁₋₆ alkyl, aryl-C₁₋₆ alkoxy, or aryl-C₁₋₆ alkylthio; and

R² and R³ are independently hydrogen or C₁₋₆ alkyl; and

the “*” (asterisk) in Formula 2 represents a stereogenic center.

Another aspect of the present invention provides a method of preparing compounds represented by Formula 2, above, the method comprising reacting a compound of Formula 1, 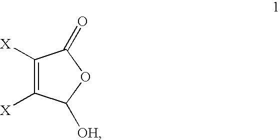
with a compound of Formula 3, 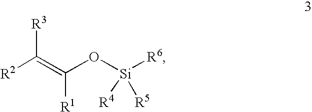
in the presence of a Lewis acid catalyst to yield the compound of Formula 2, wherein X in Formula 1 and R¹, R², and R³ in Formula 3 are as defined in Formula 2 above, and R⁴, R⁵, and R⁶ in Formula 3 are independently C₁₋₆ alkyl.

A further aspect of the present invention provides a method of making 2,3-dihalo-4-(2-oxo-furan-5-yl)-buten-2-olide, the method comprising reacting a mucohalic acid with (furan-2-yloxy)-trimethyl-silane in the presence of a Lewis acid and solvent.

An additional aspect of the present invention provides a 2,3-dihalo-4-(2-oxo-furan-5-yl)-buten-2-olide.

### DETAILED DESCRIPTION

### DEFINITIONS AND ABBREVIATIONS

Unless otherwise indicated, this disclosure uses definitions provided below. Some of the definitions and formulae may include a “-” (dash) to indicate a bond between atoms or a point of attachment to a named or unnamed atom or group of atoms. Other definitions and formulae may include an “=” (equal sign) or “≡” (identity sign) to indicate a double bond or a triple bond, respectively. Certain formulae may also include an “*” (asterisk) to indicate a stereogenic (chiral) center. Such formulae may refer to the racemate or to samples of individual enantiomers, which may or may not be substantially enantiomerically pure.

“Substituted” groups are those in which one or more hydrogen atoms have been replaced with one or more non-hydrogen groups, provided that valence requirements are met and that a chemically stable compound results from the substitution.

“About” or “approximately,” when used in connection with a measurable numerical variable, refers to the indicated value of the variable and to all values of the variable that are within the experimental error of the indicated value (e.g., within the 95% confidence interval for the mean) or within ±10 percent of the indicated value, whichever is greater.

“Alkyl” refers to straight chain and branched saturated hydrocarbon groups, generally having a specified number of carbon atoms (i.e., C₁₋₆ alkyl refers to an alkyl group having 1, 2, 3, 4, 5, or 6 carbon atoms. Examples of alkyl groups include, without limitation, methyl, ethyl, n-propyl, i-propyl, n-butyl, s-butyl, i-butyl, t-butyl, pent-1-yl, pent-2-yl, pent-3-yl, 3-methylbut-1-yl, 3-methylbut-2-yl, 2-methylbut-2-yl, 2,2,2-trimethyleth-1-yl, n-hexyl, and the like.

“Alkenyl” refers to straight chain and branched hydrocarbon groups having one or more unsaturated carbon-carbon bonds, and generally having a specified number of carbon atoms. Examples of alkenyl groups include, without limitation, ethenyl, 1-propen-1-yl, 1-propen-2-yl, 2-propen-1-yl, 1-buten-1-yl, 1-buten-2-yl, 3-buten-1-yl, 3-buten-2-yl, 2-buten-1-yl, 2-buten-2-yl, 2-methyl-i-propen-1-yl, 2-methyl-2-propen-1-yl, 1,3-butadien-1-yl, 1,3-butadien-2-yl, and the like.

“Alkynyl” refers to straight chain or branched hydrocarbon groups having one or more triple carbon-carbon bonds, and generally having a specified number of carbon atoms. Examples of alkynyl groups include, without limitation, ethynyl, 1-propyn-1-yl, 2-propyn-1-yl, 1-butyn-1-yl, 3-butyn-1-yl, 3-butyn-2-yl, 2-butyn-1-yl, and the like.

“Alkanoyl,” “alkanoyloxy,” and “alkanoylamino” refer, respectively, to alkyl-C(O)—, alkyl-C(O)—O—, and alkyl-C(O)—NH—, where alkyl is defined above, and generally includes a specified number of carbon atoms, including the carbonyl carbon. Examples of alkanoyl groups include, without limitation, formyl, acetyl, propionyl, butyryl, pentanoyl, hexanoyl, and the like.

“Alkoxy,” “alkoxycarbonyl,” “alkoxycarbonyloxy,” and “alkoxycarbonylamino” refer, respectively, to alkyl-O—, alkyl-O—C(O)—, alkyl-O—C(O)—O—, and alkyl-O—C(O)—NH—, where alkyl is defined above. Examples of alkoxy groups include, without limitation, methoxy, ethoxy, n-propoxy, i-propoxy, n-butoxy, s-butoxy, t-butoxy, n-pentoxy, s-pentoxy, and the like.

“Alkylamino,” “alkylaminocarbonyl,” “dialkylaminocarbonyl,” “alkylsulfonyl,” “sulfonylaminoalkyl,” “alkylsulfonylaminocarbonyl,” and “alkylthio” refer, respectively, to alkyl-NH—, alkyl-NH—C(O)—, alkyl₂—N—C(O)—, alkyl-S(O₂)—, HS(O₂)—NH-alkyl-, alkyl-S(O)—NH—C(O)—, and alkyl-S—, where alkyl is defined above.

“Aminoalkyl” and “cyanoalkyl” refer, respectively, to NH₂—alkyl and N—C—alkyl, where alkyl is defined above.

“Cycloalkyl” refers to saturated monocyclic and bicyclic hydrocarbon rings, generally having a specified number of carbon atoms that comprise the ring (i.e., C₃₋₇ cycloalkyl refers to a cycloalkyl group having 3, 4, 5, 6 or 7 carbon atoms as ring members and C₃₋₁₂ cycloalkyl refers to a cycloalkyl group having 3, 4, 5, 6, 7, 8, 9, 10, 11, or 12 carbon atoms as ring members). Cycloalkyl groups may be attached to a parent group or to a substrate at any ring atom, unless such attachment would violate valence requirements. Likewise, the cycloalkyl group may include one or more non-hydrogen substituents unless such substitution would violate valence requirements. Useful substituents include, without limitation, alkyl, alkoxy, alkoxycarbonyl, and alkanoyl, as defined above, and hydroxy, mercapto, nitro, halogen, and amino.

Examples of monocyclic cycloalkyl groups include, without limitation, cyclopropyl, cyclobutyl, cyclopentyl, cyclohexyl, and the like. Examples of bicyclic cycloalkyl groups include, without limitation, bicyclo[1.1.0]butyl, bicyclo[1.1.1]pentyl, bicyclo[2.1.0]pentyl, bicyclo[2.1.1]hexyl, bicyclo[3.1.0]hexyl, bicyclo[2.2.1]heptyl, bicyclo[3.2.0]heptyl, bicyclo[3.1.1]heptyl, bicyclo[4.1.0]heptyl, bicyclo[2.2.2]octyl, bicyclo[3.2.1]octyl, bicyclo[4.1.1]octyl, bicyclo[3.3.0]octyl, bicyclo[4.2.0]octyl, bicyclo[3.3.1]nonyl, bicyclo[4.2.1]nonyl, bicyclo[4.3.0]nonyl, bicyclo[3.3.2]decyl, bicyclo[4.2.2]decyl, bicyclo[4.3.1]decyl, bicyclo[4.4.0]decyl, bicyclo[3.3.3]undecyl, bicyclo[4.3.2]undecyl, bicyclo[4.3.3]dodecyl, and the like, which may be attached to a parent group or substrate at any of the ring atoms, unless such attachment would violate valence requirements.

“Cycloalkanoyl” refers to cycloalkyl-C(O)—, where cycloalkyl is defined above, and generally includes a specified number of carbon atoms, excluding the carbonyl carbon. Examples of cycloalkanoyl groups include, without limitation, cyclopropanoyl, cyclobutanoyl, cyclopentanoyl, cyclohexanoyl, cycloheptanoyl, and the like.

“Halo,” “halogen” and “halogeno” may be used interchangeably, and refer to fluoro, chloro, bromo, and iodo.

“Haloalkyl” and “haloalkanoyl” refer, respectively, to alkyl or alkanoyl groups substituted with one or more halogen atoms, where alkyl and alkanoyl are defined above. Examples of haloalkyl and haloalkanoyl groups include, without limitation, trifluoromethyl, trichloromethyl, pentafluoroethyl, pentachloroethyl, trifluoroacetyl, trichloroacetyl, pentafluoropropionyl, pentachloropropionyl, and the like.

“Hydroxyalkyl” and “oxoalkyl” refer, respectively, to HO—alkyl and O═alkyl, where alkyl is defined above. Examples of hydroxyalkyl and oxoalkyl groups, include, without limitation, hydroxymethyl, hydroxyethyl, 3-hydroxypropyl, oxomethyl, oxoethyl, 3-oxopropyl, and the like.

“Aryl” and “arylene” refer to monovalent and divalent aromatic groups, respectively. Examples of aryl groups include, without limitation, phenyl, naphthyl, biphenyl, pyrenyl, anthracenyl, fluorenyl, and the like, which may be unsubstituted or substituted with 1 to 4 substituents. Such substituents include, without limitation, alkyl, alkoxy, alkoxycarbonyl, alkanoyl, and cycloalkanoyl, as defined above, and hydroxy, mercapto, nitro, halogen, and amino.

“Arylalkyl” refers to aryl-alkyl, where aryl and alkyl are defined above. Examples include, without limitation, benzyl, fluorenylmethyl, and the like.

“Arylalkanoyl” refers to aryl-alkanoyl, where aryl and alkanoyl are defined above. Examples include, without limitation, benzoyl, phenylethanoyl, phenylpropanoyl, and the like.

“Arylalkoxycarbonyl” refers to aryl-alkoxycarbonyl, where aryl and alkoxycarbonyl are defined above. Examples include, without limitation, phenoxycarbonyl, benzyloxycarbonyl (CBz), and the like.

“Heterocycle” and “heterocyclyl” refer to saturated, partially unsaturated, or unsaturated monocyclic or bicyclic rings having from 5 to 7 or from 7 to 11 ring members, respectively. These groups have ring members made up of carbon atoms and from 1 to 4 heteroatoms that are independently nitrogen, oxygen or sulfur, and may include any bicyclic group in which any of the above-defined monocyclic heterocycles are fused to a benzene ring. The nitrogen and sulfur heteroatoms may optionally be oxidized. The heterocyclic ring may be attached to a parent group or to a substrate at any heteroatom or carbon atom unless such attachment would violate valence requirements. Likewise, any of the carbon or nitrogen ring members may include a non-hydrogen substituent unless such substitution would violate valence requirements. Useful substituents include, without limitation, alkyl, alkoxy, alkoxycarbonyl, alkanoyl, and cycloalkanoyl, as defined above, and hydroxy, mercapto, nitro, halogen, and amino.

Examples of heterocycles include, without limitation, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H, 6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyriridinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

“Heteroaryl” and “heteroarylene” refer, respectively, to monovalent and divalent heterocycles or heterocyclyl groups, as defined above, which are aromatic. Heteroaryl and heteroarylene groups represent a subset of aryl and arylene groups, respectively.

“Enantiomeric excess” or “ee” is a measure, for a given sample, of the excess of one enantiomer over a racemic sample of a chiral compound and is expressed as a percentage. Enantiomeric excess is defined as 100×(er−1)/(er+1), where “er” is the ratio of the more abundant enantiomer to the less abundant enantiomer.

“Enantioselectivity” or variants thereof refers to a given reaction or chemical transformation (e.g., ester hydrolysis, hydrogenation, hydroformnylation, π-allyl palladium coupling, hydrosilation, hydrocyanation, olefin metathesis, hydroacylation, allylamine isomerization, aldol addition, etc.) that yields more of one enantiomer than another.

“High level of enantioselectivity” refers to a given reaction that yields product with an ee of at least about 80%.

“Enantiomerically enriched” refers to a sample of a chiral compound, which has more of one enantiomer than another. The degree of enrichment is measured by er or ee.

“Substantially pure enantiomer” or “substantially enantiopure” refers to a sample of an enantiomer having an ee of about 90% or greater.

“Enantiomerically pure” or “enantiopure” refers to a sample of an enantiomer having an ee of about 99% or greater.

“Opposite enantiomer” refers to a molecule that is a non-superimposable mirror image of a reference molecule, which may be obtained by inverting all of the stereogenic centers of the reference molecule. For example, if the reference molecule has S absolute stereochemical configuration, then the opposite enantiomer has R absolute stereochemical configuration. Likewise, if the reference molecule has S,S absolute stereochemical configuration, then the opposite enantiomer has R,R stereochemical configuration, and so on.

“Lewis acid catalyst” refers to an electrophilic compound that promotes a given reaction and comprises a central atom, such as Ti, Zn, etc., which lacks a full valence shell of electrons, and as a result, can accept a lone pair of electrons, in the form of a bond, from an electron-rich species in order to fill up its valence shell. The Lewis acid catalyst may be chiral or achiral.

Table 1 lists abbreviations, which are used throughout the specification.

TABLE 1
List of Abbreviations

| Abbreviation | Description |
|---|---|
| Ac | acetyl |
| ACN | acetonitrile |
| Aq | aqueous |
| BINOL | [1,1′]binaphthalenyl-2,2′-diol |
| Bn | benzyl |
| Bu | butyl |
| t-Bu | tertiary butyl |
| t-BuOK | potassium tertiary butoxide |
| CO₂Me | methoxycarbonyloxy |
| CO₂t-Bu | tertiarybutoxycarbonyloxy |
| COMe | methylcarbonyl (acetyl) |
| Cu(II)-box | 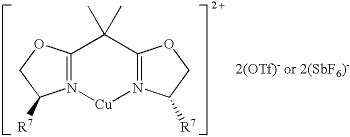 |
| | where R⁷ is t-Bu, i-Pr, Ph, or Bn |
| Cu(II)-pybox | 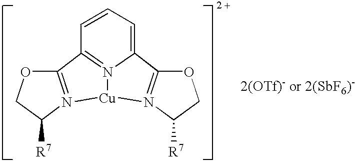 |
| | where R⁷ is t-Bu, i-Pr, Ph, or Bn |
| DIBAL | diisobutylaluminum hydride |
| DME | dimethyl ether |
| DMF | dimethylformamide |
| DMSO | dimethylsulfoxide |
| Et | ethyl |
| ET₃N | triethylamine |
| EtOH | ethyl alcohol |
| Et₂O | ethyl ether |
| EtOAc | ethyl acetate |
| h, min, s, d | hours, minutes, seconds, days |
| LDA | lithium diisopropylamide |
| LiHMDS | lithium hexamethyldisilazide |
| Me | methyl |
| MeOH | methyl alcohol |
| Me₃SiCl | chloro-trimethyl-silane |
| NaOAc | sodium acetate |
| NH₄OAc | ammonium acetate |
| NMP | N-methylpyrrolidone |
| NR | no reaction |
| 3-OCH₂O-4 | methylenedioxy |
| p-OMe | para-methoxy |
| PdCl₂(dppf)₂ | dichloro[1,1′-bis(diphenylphosphino)ferrocene]palladium |
| | (II) dichloromethane adduct |
| Pd₂(dba)₃ | tris(dibenzylidene-acetone)dipalladium(0) |
| Pd(PPh₃)₄ | tetrakis(triphenylphosphine)palladium(0) |
| Ph | phenyl |
| Ph₃P | triphenylphosphine |
| Pr | propyl |
| ppm | parts per million |
| i-Pr | isopropyl |
| i-PrOH | isopropyl alcohol |
| RT | room temperature (approximately 20° C. to 25° C.) |
| Tf | trifluoromethanesulfonyl or triflyl |
| TFA | trifluoroacetic acid |
| THF | tetrahydrofuran |
| TLC | thin-layer chromatography |
| TMS | trimethylsilyl |

In some of the reaction schemes and examples below, certain compounds can be prepared using protecting groups, which prevent undesirable chemical reaction at otherwise reactive sites. Protecting groups may also be used to enhance solubility or otherwise modify physical properties of a compound. For a discussion of protecting group strategies, a description of materials and methods for installing and removing protecting groups, and a compilation of useful protecting groups for common functional groups, including amines, carboxylic acids, alcohols, ketones, aldehydes, and the like, see T. W. Greene and P. G. Wuts, Protecting Groups in Organic Chemistry (1999) and P. Kocienski, Protective Groups (2000), which are herein incorporated by reference in their entirety for all purposes.

In addition, some of the schemes and examples below may omit details of common reactions, including oxidations, reductions, and so on, which are known to persons of ordinary skill in the art of organic chemistry. The details of such reactions can be found in a number of treatises, including Richard Larock, Comprehensive Organic Transformations (1999), and the multi-volume series edited by Michael B. Smith and others, Compendium of Organic Synthetic Methods (1974-2003). Starting materials and reagents may be obtained from commercial sources or may be prepared from literature sources.

Generally, the chemical transformations described throughout the specification may be carried out using substantially stoichiometric amounts of reactants, though certain reactions may benefit from using an excess of one or more of the reactants. Additionally, many of the reactions disclosed throughout the specification, may be carried out at about RT, but particular reactions may require the use of higher or lower temperatures, depending on reaction kinetics, yields, and the like. In this regard, any references in the disclosure to a concentration range, a temperature range, a pH range, a catalyst loading range, and so on, whether expressly using the word “range” or not, includes the indicated endpoints.

Many of the chemical transformations may employ one or more compatible solvents, which may influence the reaction rate and yield. Depending on the nature of the reactants, the one or more solvents may be polar protic solvents, polar aprotic solvents, non-polar solvents, or some combination.

Scheme I shows a method of making 4-substituted-2-buten-4-olides (Formula 2). The method includes reacting a mucohalic acid (Formula 1) with a silyl enol ether (Formula 3) in the presence of a Lewis acid and solvent to give a 4-substituted-2-buten-4-olide (Formula 2). In Formula 1-3, X is halogen (Cl, Br, or I), R¹ is C₁₋₆ alkyl, C₃₋₈ cycloalkyl, C₁₋₆ alkoxy, C₁₋₆ alkylthio, aryl, aryl-C₁₋₆ alkyl, aryl-C₁₋₆ alkoxy, or aryl-C₁₋₆ alkylthio, and R² and R³ are independently hydrogen or C₁₋₆ alkyl, and R⁴, R⁵, and R⁶ are independently C₁₋₆ alkyl. Particularly useful X substituents include Cl and Br. Particularly useful R¹ substituents include C₁₋₆ alkoxy, C¹⁻⁶ alkylthio, and aryl substituents, including phenyl groups having zero to four, non-hydrogen substituents selected from C₁₋₆ alkyl, C¹⁻⁶ alkoxy, halogen, hydroxy, mercapto, oxy, nitro, halogen, or amino. Especially useful R⁴, R⁵, and R⁶ include Me.

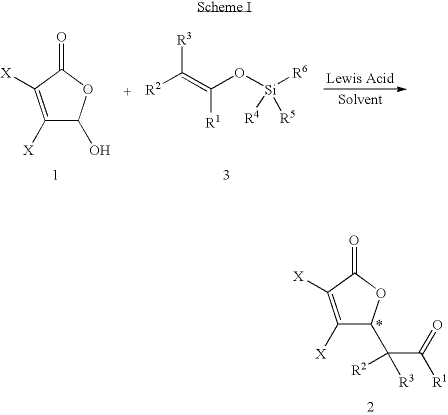

As noted in the examples below, the conversion of mucohalic acids (Formula 1) to 4-substituted-2-buten-4-olides (Formula 2) may depend on the choice of Lewis acid catalyst and solvent. For instance, the aldol reaction may be carried out in the presence of a chiral Lewis acid to produce an enantiomerically enriched or enantiopure product (Formula 2). In other cases, the aldol reaction may be carried out in the presence of an achiral Lewis acid, which will generate a racemic product (Formula 2). In any event, a wide variety of Lewis acids and solvents may be used. Useful solvents include, without limitation, aprotic solvents, such as 1,4-dioxane, THF, Et₂O, DME, dichloromethane, trichloromethane, dichloroethane, nitromethane, ACN, NMP, DMF, DMSO, toluene, and the like.

As noted above, the Lewis acid may be achiral or chiral. Achiral Lewis acids may include compounds having the formula MXₙ, where M is Al, As, B, Fe, Ga, Mg, Nb, Sb, Sn, Ti, and Zn, X is a halogen, and n is an integer from 2 to 5, inclusive, depending on the valence state of M. Examples of compounds of formula MXₙ include, but are not limited to, AlCl₃, AlI₃, AlF₃, AlBr₃, AsCl₃, AsI₃, AsF₃, AsBr_{3 BCl}₃, BBr₃, BI₃, BF₃, FeCl₃, FeBr₃, FeI₃, FeF₃, FeCl₂, FeBr₂, FeI₂ , FeF₂, GaGl₃, GaI₃, GaF₃, GaBr₃, MgCl₂, MgI₂, MgF₂, MgBr₂, NbCl₅, SbCl₃, SbI₃, SbF₃, SbBr₃, SbCl₅, SbI₅, SbF₅, SbBr₅, SnCl₂, SnI₂, SnF₂, SnBr₂, SnCl₄, SnI_{4,} SnF₄, SnBr₄ , TiBr₄, TiCl₂, TiCl₃, TiCl₄, TiF₃, TiF₄, TiI_{4,} ZnCl₂, ZnI₂, ZnF₂, and ZnBr₂. Other achiral Lewis acids, include, but are not limited to, Al₂O₃, BF₃BCl₃·SMe₂, BI₃·SMe₂, BF₃·SMe₂, BBr₃·SMe₂, BF₃·OEt₂, Et₂AlCl, EtAlCl₂, MgCl₂·OEt₂, MgI₂·OEt₂, MgF₂·OEt₂, MgBr₂·OEt₂, Et₂AlCl, EtAlCl₂, LiClO₄, Ti(O-i-Pr)₄, and Zn(OAc)₂. Still other achiral Lewis acids include, but are not limited to, salts of Cobalt (II), Copper (II), and Nickel (II), such as (CH₃CO₂)₂Co, CoBr₂, CoCl₂, CoF₂, CoI₂, Co(NO₃)₂, cobalt (II) triflate, cobalt (II) tosylate, (CH₃CO₂)₂Cu, CuBr₂, CuCl₂, CuF₂, CuI₂, Cu(NO₃)₂, copper (II) triflate, copper (II) tosylate, (CH₃CO₂)₂Ni, NiBr₂, NiCl₂, NiF₂, NiI₂, Ni(NO₃)₂, nickel (II) triflate, and nickel (II) tosylate. Monoalkyl boronhalides, dialkyl boronhalides, monoaryl boronhalides, and diaryl boronhalides may be employed as Lewis acids. In addition, rare earth metal trifluoromethansulfonates such as Eu(OTf)₃, Dy(OTf)₃, Ho(OTf)₃, Er(OTf)₃, Lu(OTf)₃, Yb(OTf)₃, Nd(OTf)₃, Gd(OTf)₃, Lu(OTf)₃, La(OTf)₃, Pr(OTf)₃, Tm(OTf)₃, Sc(OTf)₃, Sm(OTf)₃, AgOTf, Y(OTf)₃, and polymer resins thereof (e.g., scandium triflate polystyrene resin, PS—Sc(OTf)₂) may be used in a solution such as one part water and four to nine parts THF. Other useful achiral Lewis acids may include, silica gels such as silica gel (CAS 112926-00-8) used for column chromatography (80-500 mesh particle size).

Examples of chiral Lewis acids include, without limitation, Sn(II) complexes modified with chiral, chelating diamine ligands, such as (S)-1-(1-methyl-pyrrolidin-2-ylmethyl)-piperidine, (S)-(1-methyl-pyrrolidin-2-ylmethyl)-naphthalen-1-yl-amine, and their opposite enantiomers. Other chiral Lewis acids include, without limitation, boron heterocycle catalysts, including (acyloxy)borane complexes, such as (R,R)-2,6-diisopropoxy-benzoic acid carboxy-(5-oxo-[1,3,2]dioxaborolan-4-yl)-methyl ester, (R,R)-2,6-diisopropoxy-benzoic acid [2-(3,5-bis-trifluoromethyl-phenyl)-5-oxo-[1,3,2]dioxaborolan-4-yl]-carboxy-methyl ester, and (R,R)-2,6-diisopropoxy-benzoic acid carboxy-[5-oxo-2-(2-phenoxy-phenyl)-[1,3,2]dioxaborolan-4-yl]-methyl ester, and oxazaborolidine catalysts, such as (R)-4-(3,4-dimethoxy-benzyl)-4-methyl-3-(toluene-4-sulfonyl)-[1,3,2]oxazaborolidin-5-one, (R,R,R)-6-isopropyl-9-methyl-1-(toluene-4-sulfonyl)-3-oxa-1-aza-2-bora-spiro[4.5]decan-4-one, (S)-4-isopropyl-3-(toluene-4-sulfonyl)-[1,3,2]oxazaborolidin-5-one, (S)-4-isopropyl-3-(4-nitro-benzenesulfonyl)-[1,3,2]oxazaborolidin-5-one, and (S)-2-butyl-4-(1H-indol-3-ylmethyl)-3-(toluene-4-sulfonyl)-[1,3,2]oxazaborolidin-5-one, including their opposite enantiomers and diastereoisomers (if applicable). Additional chiral Lewis acids include, without limitation, titanium-based complexes, including (R)— or (S)-BINOL-Ti complexes prepared by reacting (i-PrO)₂Ti(O), Cl₂Ti(Oi-Pr)₂, or Cl₂Ti(Oi-Pr)₄ with (R) or (S)—BINOL, or by reacting Cl₂Ti(Oi-Pr)₄ with a Schiff base, 2′-[(3-bromo-5-t-butyl-2-hydroxy-benzylidene)-amino]-[1,1′]binaphthalenyl-2-ol. Other useful chiral Lewis acid include, without limitation, cationic Cu(II) complexes incorporating chiral bidentate bis(oxazolinyl) (box) ligands and tridentate bis(oxazolinyl)pyridine (pybox) ligands and analogous Sn(II) complexes employing box and pybox ligands, including their opposite enantiomers and diastereoisomers. For a further discussions of chiral Lewis acids, see S. G. Nelson, Tetrahedron Asymmetry 9:357-389 (1998) and I. Ojima (ed.), Catalytic Asymmetric Synthesis 493-541 (2d ed., 2000), and references cited therein, the complete disclosures of which are herein incorporated by references for all purposes.

Particularly useful Lewis acids include, without limitation, La(OTf)₃, Mg(OTf)₂, Sc(OTf)₃, TiCl₄, ZnCl₂, Zn(OTf)₂, InCl₃, Sn(OTf)₂, BF₃·OEt₂, Pd(CF₃CO₂)₂, and the like. Other particularly useful Lewis acids include, without limitation, (R)— or (S)—BINOL—Ti complexes prepared by reacting (i-PrO)₂Ti(O), Cl₂Ti(Oi-Pr)₂, or Cl₂Ti(Oi-Pr)₄ with (R) or (S)—BINOL. Generally, catalytic amounts of the Lewis acid (e.g., from about 0.5 mol % to about 20 mol %) are sufficient to effect the transformation shown in Scheme I, though the use of one or more equivalents of the Lewis acid may be beneficial.

The conversion of mucohalic acids (Formula 1) to 4-substituted-2-buten-4-olides (Formula 2) can be undertaken using substantially stoichiometric amounts of reactants, though it may be advantageous to carryout the reaction with an excess of the silyl enol ether (e.g., from about 1.5 equivalents to about 2.5 equivalents).

As shown in the examples below, reactions between various mucohalic acids (Formula 1) and silyl enol ethers (Formula 3) at temperatures in the range of about -30° C. to about RT result in good yields of 4-substituted-2-buten-4-olides (Formula 2). Moreover, the conversions occur within a reasonable period of time (i.e., reaction times under about 24 hours). The reaction temperature may be varied from about -78° C. to about 80° C. to modify reaction time and yield, though temperature optimization appears to be catalyst dependent.

Unlike the conventional Mukaiyama aldol reaction, which generates a β-hydroxy carbonyl compound, the reaction shown in Scheme I gives predominantly γ-butenolides. Therefore, any reaction mechanism postulated for the transformation shown in Scheme I would appear to be more complicated than the reaction mechanism for the Mukaiyama aldol reaction since both carbonyl groups in mucohalic acid may be activated. Furthermore, both cyclic (Formula 1) and open (Formula 1′) forms of mucohalic acid are likely involved in the formation of the γ-butenolides.

The silyl enol ethers (Formula 3) may be obtained from commercial sources or prepared from literature methods. To make the less substituted enolate equivalent (e.g., R²═R³═H in Formula 3), one may react an appropriate ketone (Formula 4), 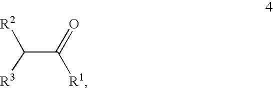
with a hindered lithium amide base (e.g., LDA, LiHMDS, etc.) in THF at −78° C. to give a kinetic lithium enolate (Formula 5), 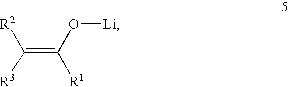
which is subsequently reacted with (R⁴R⁵R⁶)SiCl (e.g., Me₃SiCl) in THF at −78° C. to give the desired silyl enol ether (Formula 3). To make the more substituted enolate equivalent (e.g., R¹═R²═R³═C₁₋₆ alkyl), one may react the appropriate ketone (Formula 4) with (R⁴R⁵R⁶)SiCl (e.g., Me₃SiCl) at RT in the presence of a weak base (e.g., Et₃N) to give the desired silyl enol ether (Formula 3).

As discussed above, the 4-substituted-2,3-dihalo-2-buten-4-olides (Formula 2) are useful intermediates for preparing biologically active natural products and compounds. For example, Scheme II shows a method for preparing Goniothalesdiol (Formula 6) from (S)-4-benzoylmethyl-2,3-dichloro-2-buten-4-olide (Formula 7). Goniothalesdiol is a natural product that exhibits cytotoxicity against P388 mouse leukemia cells and is thought to have insecticidal activity. For a discussion of Goniothalesdiol, including a total synthesis from a chiral starting material (D-mannitol), see M. Babjak et al., Tetrahedron Letters 43:6983-85 (2002).

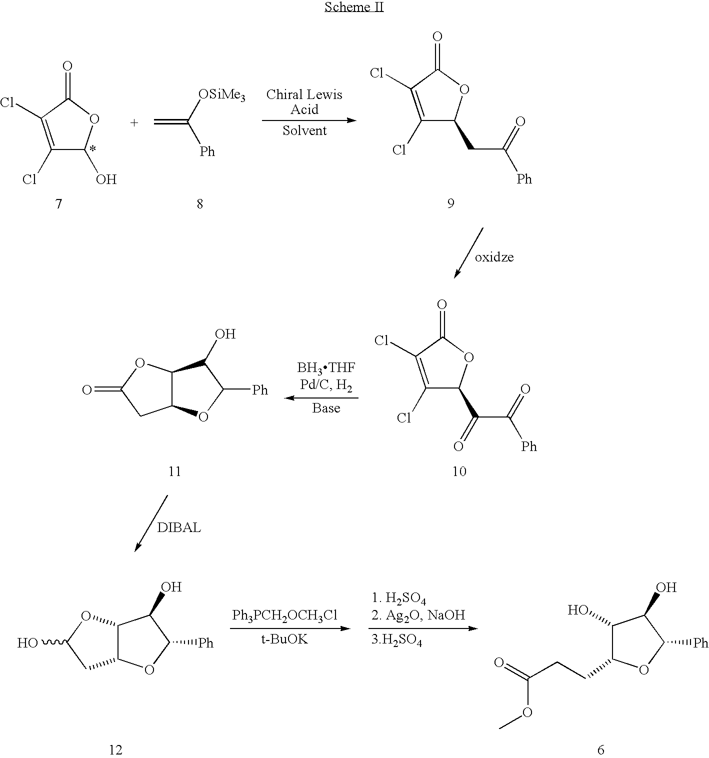

As described throughout the specification, many of the disclosed compounds have stereoisomers. Some of these compounds may exist as single enantiomers (enantiopure compounds) or mixtures of enantiomers (enriched and racemic samples), which depending on the relative excess of one enantiomer over another in a sample, may exhibit optical activity. Such stereoisomers, which are non-superimposable mirror images, possess a stereogenic axis or one or more stereogenic centers (i.e., chirality). Other disclosed compounds may be stereoisomers that are not mirror images. Such stereoisomers, which are known as diastereoisomers, may be chiral or achiral (contain no stereogenic centers). They include molecules containing an alkenyl or cyclic group, so that cisltrans (or Z/E) stereoisomers are possible, or molecules containing two or more stereogenic centers, in which inversion of a single stereogenic center generates a corresponding diastereoisomer. Unless stated or otherwise clear (e.g., through use of stereobonds, stereocenter descriptors, ee, etc.) the scope of the present invention generally includes the reference compound and its stereoisomers, whether they are each pure (e.g., enantiopure) or mixtures (e.g., enantiomerically enriched or racemic).

In addition to the asymmetric syntheses described above, individual enantiomers may be prepared, isolated, or further enriched using other techniques, such as classical resolution, chiral chromatography, or recrystallization. For example, an optically active compound may be reacted with an enantiomerically-pure compound (e.g., acid or base) to yield a pair of diastereoisomers, each composed of a single enantiomer, which are separated via, say, fractional recrystallization or chromatography. The desired enantiomer is subsequently regenerated from the appropriate diastereoisomer. Additionally, the desired enantiomer often may be further enriched by recrystallization in a suitable solvent when it is it available in sufficient quantity (e.g., typically not much less than about 85% ee, and in some cases, not much less than about 90% ee).

Some of the compounds may also contain a keto or oxime group, so that tautomerism may occur. In such cases, the present invention generally includes tautomeric forms, whether they are each pure or mixtures.

The disclosed compounds also include all isotopic variations, in which at least one atom is replaced by an atom having the same atomic number, but an atomic mass different from the atomic mass usually found in nature. Examples of isotopes suitable for inclusion in the disclosed compounds include, without limitation, isotopes of hydrogen, such as ²H and ³H; isotopes of carbon, such as ¹³C and ¹⁴C; isotopes of nitrogen, such as ¹⁵N; isotopes of oxygen, such as ¹⁷O and ¹⁸O; isotopes of phosphorus, such as ³¹P and ³²P; isotopes of sulfur, such as ³⁵S; isotopes of fluorine, such as ¹⁸F; and isotopes of chlorine, such as ³⁶Cl.

### EXAMPLES

The following examples are intended to be illustrative and non-limiting, and represent specific embodiments of the present invention.

### General Methods

All reactions were carried out under nitrogen or argon atmosphere unless otherwise noted. All solvents and reagents used were from commercial sources and no further purification was performed. Reactions were monitored by high-pressure liquid chromatography (HPLC) using either a Perkin Elmer Series 200 pump/235C diode array detector (215 nm)/Waters Symmetry C₁₈ (4.6×150 mm, 5μ)/MeCN/0.1% TFA in H₂O (60/40 isocratic) or a Perkin Elmer Series 200 System (Pump/Detector)/YMC Pack Pro Clg (4.6×150 mm, 3μ) MeCN/0.2% HClO₄ in H₂O (20/80 to 80/20 gradient) and/or mass spectrometry (MS) on a Micromass Platform LC and by thin-layer chromatography (TLC) on 0.25 mm E. Merck silica gel 60 plates (F₂₅₄) using UV light and a cerium stain as visualizing agents. E. Merck silica gel 60 (0.040-0.063 mm particle size) was used for column chromatography. Melting points were determined using a Barnstead/Thermolyne 1401 melting point apparatus in open capillaries and are uncorrected. Proton nuclear magnetic resonance (¹H NMR) spectra were recorded at 400 MHz on a Varian UNITY INOVA AS400. Chemical shifts are reported as delta (δ) units in parts per million (ppm) relative to the singlet at 7.27 ppm for CDCl₃, 3.58 ppm for THF-d₈ or 2.50 ppm for DMSO-d₆. Coupling constants (J) are reported in Hertz (Hz). Carbon-13 nuclear magnetic resonance (¹³C NMR) spectra were recorded at 100 MHz on a Varian UNITY Plus INOVA 400. Chemical shifts are reported as delta (δ) units in parts per million (ppm) relative to the center line of the triplet at 77.2 ppm for CDCl₃, the center line of the pentet at 67.6 ppm for THF-d₈ or the center line of the septet at 39.5 ppm for DMSO-d₆. Elemental analyses were performed by Quantitative Technologies Inc.

### Example 1

### Preparation of 2,3-Dichloro-4-(1-methyl-1-methoxycarbonyl-ethyl)-buten-2-olide (Scheme I, Formula 2, X═Cl, R¹=MeO, R²═R³=Me)

A solution of mucochloric acid (Formula 1, X═Cl, 169 mg, 1.0 mmol) and 0.50 M ZnCl₂ (0.20 mL, 0.10 mmol) in toluene (4 mL) was cooled to −20° C. (1-Methoxy-2-methyl-propenyloxy)-trimethyl-silane (Formula 3, R¹=MeO, R²═R³═R⁴═R⁵═R⁶=Me, 0.41 mL, 2.0 mmol) was added in one portion, the reaction solution was stirred at −20° C. for 2 h and then allowed to warm to RT over 3.5 h (0.2° C./min). It was partitioned between EtOAc (6 mL) and 50% saturated NH₄Cl (4 mL), the phases were separated and the aqueous phase was extracted with EtOAc (2 mL). The organic phases were combined, washed with H₂O (2 mL), dried (MgSO₄), and concentrated to provide crude 2,3-dichloro-4-(1-methyl-1-methoxycarbonyl-ethyl)-buten-2-olide (257 mg, 101% theory) as a colorless oil which slowly solidified. The residue was purified by SiO₂ flash chromatography [EtOAc/heptane (20/80)] to provide the titled compound (202 mg, 80% yield) as a white solid. Mp 87-88° C. ¹H NMR (CDCl₃): δ 5.37 (s, 1H), 3.76 (s, 3H), 1.37 (s, 3H), 1.13 (s, 3H). ¹³ C NMR (CDCl₃): δ 174.0, 164.9, 150.6, 122.8, 85.4, 52.8, 46.0, 23.0, 18.0. Anal. Calc'd for C₉H₁₀Cl₂O₄ (253.08): C, 42.71; H, 3.98; Cl, 28.02. Found: C, 42.96; H, 3.83; Cl, 27.80.

### EXAMPLE 2

### Preparation of 2,3-Dibromo-4-(1-methyl-1-methoxycarbonyl-ethyl)-buten-2-olide (Scheme I, Formula 2, X═Br, R¹=MeO, R²═R³=Me)

A solution of mucobromic acid (Formula 1, X═Br, 258 mg, 1.0 mmol) and 0.50 M ZnCl₂ (0.20 mL, 0.10 mmol) in toluene (4 mL) was cooled to −20° C. (1-Methoxy-2-methyl-propenyloxy)-trimethyl-silane (Formula 3, R¹=MeO, R²═R³═R⁴═R⁵═R⁶=Me, 0.41 mL, 2.0 mmol) was added in one portion; the reaction solution was stirred at +20° C. for 2 h, allowed to warm to 18° C. over 3 h (0.2° C/min) and stirred at 18° C. for 16 h. The solution was then partitioned between EtOAc (6 mL) and 50% saturated NH4Cl (4 mL). The phases were separated; the organic phase was washed with H₂O (2 mL), dried (MgSO₄), and concentrated to provide crude methyl 2,3-dibromo-4-(1-methyl-1-methoxycarbonyl-ethyl)-buten-2-olide (375 mg, 110% theory) as a colorless oil which slowly solidified. The residue was purified by SiO₂ flash chromatography [EtOAc/heptane (20/80)] to provide the titled compound (257 mg, 75% yield) as a white solid. Mp 112-113° C. ¹H NMR (CDCl₃): δ 5.39 (s, 1H), 3.76 (s, 3H), 1.38 (s, 3H), 1.10 (s, 3H). 1³C NMR (CDCl₃): δ 174.2, 165.8, 145.5, 116.8, 87.9, 52.8, 46.1, 23.5, 17.7. Anal. Calc'd for C₉H₁₀Br₂O₄ (342.00): C, 31.61; H, 2.95; Br, 46.73. Found: C, 31.78; H, 2.70; Br, 46.51.

### Example 3

### Preparation of 2,3-Dichloro-4-(2-oxo-furan-5-yl)-buten-2-olide

A solution of mucochloric acid (Formula 1, X═Cl, 169 mg, 1.0 mmol) and 0.50 M ZnCl₂ (0.20 mL, 0.10 mmol) in toluene (4 mL) was cooled to −20° C. (Furan-2-yloxy)-trimethyl-silane was added in one portion, the reaction solution was stirred at −20° C. for 2 h, allowed to warm to RT over 3.5 h (0.2° C./min) and stirred at RT for 3 d. The reaction solution was partitioned between EtOAc (6 mL) and 50% saturated NH₄Cl (4 mL); the phases were separated and the aqueous phase was extracted with EtOAc (2 mL). The organic phases were combined, washed with H₂O (2 mL), dried (MgSO₄), and concentrated to provide crude 2,3-dichloro-4-(2-oxo-furan-5-yl)-buten-2-olide (252 mg, 107% theory) as a light yellow oil which slowly solidified. HPLC analysis showed 90/10 syn/anti mixture of isomers. The residue was triturated in CH₂Cl₂ (2 mL) and the supernatant was decanted. The solid was washed with CH₂Cl₂ (1 mL), air-dried, and finally dried in vacuo to provide the titled compound (76 mg, 32% yield) as a white solid. The supernatant and wash liquids were combined, concentrated, and the residue was recrystallized from MeCN/H₂O to provide a second crop of the titled compound (36 mg, 15% yield) as a white solid. Mp 162-164° C. (dec). ^{H} NMR (THF-d₈): δ 7.69 (dd, 1H, J=1.6, 5.7), 6.28 (dd, 1H, J=2.1, 5.7), 5.54 (ddd, 1H, J=1.6, 2.1, 2.2), 5.48 (d, 1H, J=2.2). ¹³C NMR (THF-d₈): δ 171.6, 164.6, 152.5, 149.9, 124.4, 122.8, 80.1, 79.5. Anal. Calc'd for C₈H₄Cl₂₀₄ (235.02): C, 40.88; H, 1.72; Cl, 30.17. Found: C, 40.90; H, 1.50; Cl, 30.11.

### Example 4

### Preparation of (1-t-Butylsulfanyl-vinyloxy)-trimethyl-silane (Formula 3, R¹=t-butylthio, R²═R³═H, R⁴═R⁵═R⁶=Me)

A solution of 1.8 M LDA (41 mL, 73.6 mmol) in heptane/THF/ethyl-benzene was diluted in THF (30 mL) and cooled to −65° C. To the cold solution was added, drop wise, S-t-butyl thioacetate (Formula 4, R¹=t-butylthio, R²═R³═H, 10.0 mL, 70.1 mmol) over 5 min; the reaction exothermed to −55° C. The solution was allowed to stir at −70° C. for 30 min when TMS—Cl (8.9 mL, 70.1 mmol) was added, drop wise, over 5 min; a small exotherm to −62° C. was observed. The solution was stirred for 1 h at −70° C. and then allowed to warm to RT; a white solid (LiCl) precipitated. The reaction mixture was partitioned between ice-H₂O (100 mL) and heptane (100 mL). The biphasic mixture was separated and the aqueous phase was extracted with heptane (100 mL). The organic phases were combined, dried (MgSO₄) and concentrated (40° C./7 Torr) to provide the titled compound (9.2 g, 64% yield) as a pale yellow liquid; this was used without further purification. Note: a moderate loss of product (ca. 27%) occurred because (1-t-Butylsulfanyl-vinyloxy)-trimethyl-silane began to distill under these conditions. ¹H NMR (CDCl₃): δ 4.69 (d, 1H, J=0.5), 4.60 (d, 1H, J=0.5), 1.39 (s, 9H), 0.26 (s, 9H).

### Example 5

### Preparation of 2,3-Dichloro-4-(t-butyl-thio-carbonylmethyl)-buten-2-olide (Scheme I, Formula 2, X═Cl, R¹=t-butylthio, R²═R³═H)

A solution of mucochloric acid (Formula 1, X═Cl, 169 mg, 1.0 mmol) and 0.50 M ZnCl₂ (0.20 mL, 0.10 mmol) in toluene (4 mL) was cooled to −20° C. (1 -t-Butylsulfanyl-vinyloxy)-trimethyl-silane (Formula 3, R¹=t-butylthio, R²═R³═H, R⁴═R⁵═R⁶=Me, 0.48 mL, 2.0 mmol) was added in one portion; the reaction solution was stirred at −20° C. for 2 h, allowed to warm to RT over 3.5 h (0.2° C./min) and stirred at RT for 3 d. The mixture was partitioned between EtOAc (6 mL) and 50% saturated NH₄Cl (4 mL); the phases were separated, and the aqueous phase was extracted with EtOAc (2 mL). The organic phases were combined, washed with H₂O (2 mL), dried (MgSO₄), and concentrated to provide crude 2,3-dichloro-4-(t-butyl-thio-carbonylmethyl)-buten-2-olide (317 mg, 145% theory) as a brown oil. The residue was purified by SiO₂ flash chromatography [EtOAc/heptane (20/80)] to provide the titled compound (158 mg, 56% yield) as a pale yellow oil. ¹H NMR (CDCl₃): δ 5.42 (dd, 1H, J=3.9, 8.1), 3.12 (dd, 1H, J=3.9, 15.9), 2.84 (dd, 1H, J=8.1, 15.9), 1.50 (s, 9H). ¹³C NMR (CDCl₃): δ 194.1, 164.9, 151.6, 121.9, 78.4, 49.7, 45.5, 29.9. Anal. Calc'd for C₁₀H₁₂Cl₂O₃S (283.17): C, 42.41; H, 4.27; Cl, 25.04. Found: C, 42.82; H, 4.20; Cl, 22.88. KF=1.06% H₂O; HRMS (282.9957): m/e (%) 282.9962 (100.0), 284.9933 (64.0), 283.9996 (11.1), 286.9903 (10.2).

### Example 6

### Preparation of 2,3-Dichloro-4-(benzoylmethyl)-buten-2-olide (Scheme I, Formula 2, X═Cl, R¹=Ph, R²═R³═H, 1 mmol scale)

A suspension of mucochloric acid (Formula 1, X═Cl, 169 mg, 1.0 mmol) and Sc(OTf)₃ (49 mg, 0.10 mmol) in Et₂O (4 mL) was cooled to −20° C. Trimethyl-(1 -phenyl-vinyloxy)-silane (Formula 3, R¹=Ph, R²═R³═H, R⁴═R⁵═R⁶=Me, 0.41 mL, 2.0 mmol) was added in one portion. The reaction mixture was stirred at −20° C. for 1 h, allowed to warm to 15° C. over 3 h (0.2° C./min) and stirred at 15° C. for 16 h. The reaction mixture was partitioned between EtOAc (6 mL) and 50% saturated NH₄Cl (4 mL) and the phases were separated. The organic phase was washed with H₂O (2 mL), dried (MgSO₄) and concentrated to provide crude 2,3-dichloro-4-(benzoylmethyl)-buten-2-olide (286 mg, 105% theory) as a white solid. The residue was triturated in Et₂O (4 mL) and the supernatant was decanted. The remaining solid was washed with Et₂O (2×0.5 mL), air-dried and dried further in vacuo to provide the titled compound (186 mg, 69% yield) as a white solid. The supernatant and washings were combined, concentrated, and the purification process was repeated to provide a second crop of the titled compound (27 mg, 10% yield) as a white solid. Mp 121-122° C. ¹H NMR (CDCl₃): δ 7.92 (m, 2H), 7.62 (m, 1 H), 7.49 (m, 2H), 5.72 (dd, 1H, J=3.7, 8.1), 3.54 (dd, 1H, J=3.7, 17.6), 3.40 (dd, 1H, J=8.1, 17.6). ¹³C NMR (CDCl₃): δ 193.9, 165.1, 152.2, 136.0, 134.4, 129.2, 128.4, 121.7, 78.3, 40.4. Anal. Calc'd for Cl₂H₈Cl₂O₃ (271.10): C, 53.17; H, 2.97; Cl, 26.15. Found: C, 53.20; H, 2.83; Cl, 26.04.

### Example 7

### Preparation of 2,3-Dichloro-4-(benzoylmethyl)-buten-2-olide (Scheme I, Formula 2, X═Cl, R¹=Ph, R²═R³═H, 35 mmol scale)

A solution of trimethyl-(1-phenyl-vinyloxy)-silane (Formula 3, R¹=Ph, R²═R³═H, R⁴═R⁵═R⁶=Me, 14.6 mL, 71 mmol) in Et₂O (60 mL) was cooled to −20° C. To the cold solution was added Sc(OTf)₃ (0.9 g, 5 mol %) followed by the portion-wise addition of mucochloric acid (Formula 1, X═Cl, 6.0 g, 35.5 mmol) over 10 min. The reaction mixture was stirred at −20° C. for 1 h, warmed to RT over 1 h and stirred at RT for 16 h. The reaction mixture was partitioned between EtOAc (100 mL) and 50% saturated NH₄Cl (50 mL). The phases were separated and the organic phase was washed with H₂O (50 mL), brine (40 mL), was dried (MgSO₄) and concentrated to provide a tan solid (contaminated with acetophenone). The solid was triturated in Et₂O (40 mL) and collected by filtration. The filter-cake was washed with Et₂O (2×20 mL), air-dried and dried further in vacuo to provide the titled compound (7.58 g, 79% yield) as an off-white powder.

### Example 8

### Preparation 2,3-Dibromo-4-(benzoylmethyl)-buten-2-olide (Scheme I, Formula2,X═Br, R¹=Ph, R²═R³═H)

A suspension of mucobromic acid (Formula 1, X ═Br, 258 mg, 1.0 mmol) and Sc(OTf)₃ (49 mg, 0.10 mmol) in Et₂O (4 mL) was cooled to −20° C. Trimethyl-(1 -phenyl-vinyloxy)-silane (Formula 3, R¹=Ph, R²═R³═H, R⁴═R⁵═R⁶=Me, 0.41 mL, 2.0 mmol) was added in one portion. The reaction mixture was stirred at −20° C. for 2 h, allowed to warm to 18° C. over 3 h (0.2° C./min) and stirred at 18° C. for 16 h. The mixture was partitioned between warm EtOAc (15 mL) and 50% saturated NH₄Cl (4 mL) and the phases were separated. The organic phase was washed with H₂O (2 mL), dried (MgSO₄) and concentrated to provide crude 2,3-dibromo-4-(benzoylmethyl)-buten-2-olide (385 mg, 107% theory) as a white solid. The solid was triturated in Et₂O (4 mL) and collected by filtration. The filter-cake was washed with Et₂O (2×1 mL), air-dried and dried further in vacuo to provide the titled compound (264 mg, 73% yield) as a white solid. Mp 168-169° C. (dec). ¹H NMR (CDCl₃): δ 7.93 (m, 2H), 7.61 (m, 1 H), 7.48 (m, 2H), 5.72 (dd, 1H, J=3.4, 8.3(dd, 1H, J=3.4, 17.6), 3.39 (dd, 1H, J=8.3, 17.6). ¹³C NMR (CDCl₃): δ 194.0, 165.9, 147.7, 136.1, 134.3, 129.1, 128.4, 115.6, 81.1, 40.8. Anal. Calc'd for C₁₂H₈Br₂O₃ (357.88): C, 40.04; H, 2.24; Br, 44.39. Found: C, 40.43; H, 2.09; Br, 44.22.

### Example 9-30

### Preparation of 4-substituted-2,3-dichloro-2-buten-4-olides (Scheme III, Formula 13)-Effect of Lewis Acid Catalyst on Yield

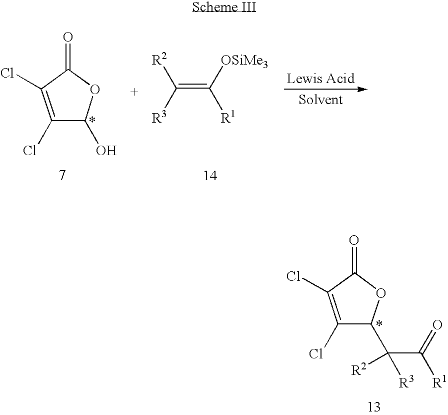

Table 2 lists Lewis acid catalyst, solvent, and yields for the preparation of 4-substituted-2,3-dichloro-2-buten-4-olides (Formula 13) via reaction of mucochloric acid (Formula 7) with various silyl enol ethers (Formula 14). For each of the entries in Table 2, a solution of mucochloric acid (Formula 7, 0.25M, 1.0 mmol) and a Lewis acid (0.10 mol) in solvent was cooled to −20° C. A silyl enol ether (Formula 14, 2.0 mmol) was added in one portion and the reaction mixture was stirred at −20° C. for 2 h and then allowed to warm to RT, with stirring, over 3 h (0.2° C./min). The yield was determined by HPLC (215 nm).

TABLE 2
Preparation of 4-substituted-2,3-dichloro-2-buten-4-olides
(Formula 13) via reaction of mucochloric acid (Formula
7) with various silyl enol ethers (Formula 14)

| | | | | Lewis Acid | | |
|---|---|---|---|---|---|---|
| Example | R¹ | R² | R³ | Catalyst | Solvent | Yield |
| 9 | MeO | Me | Me | La(OTf)₃ | Toluene | 3 |
| 10 | MeO | Me | Me | Mg(OTf)₂ | Toluene | 1 |
| 11 | MeO | Me | Me | Sc(OTf)₃ | Toluene | 84 |
| 12 | MeO | Me | Me | TiCl₄ | Toluene | 58 |
| 13 | MeO | Me | Me | ZnCl₂ | Toluene | 88 |
| 14 | MeO | Me | Me | Zn(OTf)₂ | Toluene | 52 |
| 15 | MeO | Me | Me | InCl₃ | Toluene | 7 |
| 16 | MeO | Me | Me | Sn(OTf)₂ | Toluene | 70 |
| 17 | MeO | Me | Me | BF₃.OEt₂ | Toluene | 17 |
| 18 | MeO | Me | Me | None | Toluene | — |
| 19 | MeO | Me | Me | Pd(CF₃CO₂)₂ | Toluene | 1 |
| 20 | Ph | H | H | La(OTf)₃ | Et₂O | — |
| 21 | Ph | H | H | Mg(OTf)₂ | Et₂O | — |
| 22 | Ph | H | H | Sc(OTf)₃ | Et₂O | 79 |
| 23 | Ph | H | H | TiCl₄ | Et₂O | 37 |
| 24 | Ph | H | H | ZnCl₂ | Et₂O | 43 |
| 25 | Ph | H | H | Zn(OTf)₂ | Et₂O | 4 |
| 26 | Ph | H | H | InCl₃ | Et₂O | 64 |
| 27 | Ph | H | H | Sn(OTf)₂ | Et₂O | 61 |
| 28 | Ph | H | H | BF₃.OEt₂ | Et₂O | 16 |
| 28 | Ph | H | H | None | Et₂O | — |
| 30 | Ph | H | H | Pd(CF₃CO₂)₂ | Et₂O | — |

### Example 31-40

### Preparation of 4-substituted-2,3-dichloro-2-buten-4-olides (Scheme III, Formula 13)-Effect of Temperature on Yield

Table 3 lists reaction temperature and yields for the preparation of 4-substituted-2,3-dichloro-2-buten-4-olides (Formula 13) via reaction of mucochloric acid (Formula 7) with various silyl enol ethers (Formula 14). For each of the entries in Table 3, a solution of mucochloric acid (Formula 7, 0.25M, 1.0 mmol) and a Lewis acid (0.10 mmol) in CH₂Cl₂ was cooled to the reaction temperature. A silyl enol ether (Formula 14, 2.0 mmol) was added in one portion and the reaction mixture was stirred at the reaction temperature for 16 h. The yield was determined by HPLC.

TABLE 3
Preparation of 4-substituted-2,3-dichloro-2-buten-4-olides
(Formula 13) via reaction of mucochloric acid (Formula
7) with various silyl enol ethers (Formula 14)

| | | | | Lewis Acid | Temperature | |
|---|---|---|---|---|---|---|
| Example | R¹ | R² | R³ | Catalyst | ° C. | Yield |
| 31 | MeO | Me | Me | ZnCl₂ | −30 | 94 |
| 32 | MeO | Me | Me | ZnCl₂ | −10 | 94 |
| 33 | MeO | Me | Me | ZnCl₂ | 0 | 92 |
| 34 | MeO | Me | Me | ZnCl₂ | 10 | 89 |
| 35 | MeO | Me | Me | ZnCl₂ | 20 | 84 |
| 36 | Ph | H | H | Sc(OTf)₃ | −30 | 78 |
| 37 | Ph | H | H | Sc(OTf)₃ | −10 | 78 |
| 38 | Ph | H | H | Sc(OTf)₃ | 0 | 77 |
| 39 | Ph | H | H | Sc(OTf)₃ | 10 | 74 |
| 40 | Ph | H | H | Sc(OTf)₃ | 20 | 73 |

### Example 41-56

### Preparation of 4-substituted-2,3-dichloro-2-buten-4-olides (Scheme III, Formula 13)-Effect of Solvent on Yield

Table 4 lists solvent and yields for the preparation of 4-substituted-2,3-dichloro-2-buten-4-olides (Formula 13) via reaction of mucochloric acid (Formula 7) with various silyl enol ethers (Formula 14). For each of the entries in Table 4, a solution of mucochloric acid (Formula 7, 0.25M, 1.0 mmol) and a Lewis acid catalyst (0.10 mmol) in the solvent was cooled to −20° C. A silyl enol ether (Formula 14, 2.0 mmol) as added in one portion and the reaction mixture was stirred at −20° C. for or 2 h (Example 41-48) or 4 h (Example 49-57) and then at RT for 14 h (Example 41-48) or 2 h (Example 49-57). The yield was determined by HPLC.

TABLE 4
Preparation of 4-substituted-2,3-dichloro-2-buten-4-olides
(Formula 13) via reaction of mucochloric acid (Formula
7) with various silyl enol ethers (Formula 14)

| | | | | Lewis Acid | | |
|---|---|---|---|---|---|---|
| Example | R¹ | R² | R³ | Catalyst | Solvent | Yield |
| 41 | MeO | Me | Me | ZnCl₂ | Toluene | 94 |
| 42 | MeO | Me | Me | ZnCl₂ | Et₂O | 94 |
| 43 | MeO | Me | Me | ZnCl₂ | THF | 24 |
| 44 | MeO | Me | Me | ZnCl₂ | CH₂Cl₂ | 92 |
| 45 | MeO | Me | Me | ZnCl₂ | CHCl₃ | 71 |
| 46 | MeO | Me | Me | ZnCl₂ | MeNO₂ | >96 |
| 47 | MeO | Me | Me | ZnCl₂ | EtCN | 44 |
| 48 | MeO | Me | Me | ZnCl₂ | DME | 67 |
| 49 | Ph | H | H | Sc(OTf)₃ | Toluene | 80 |
| 50 | Ph | H | H | Sc(OTf)₃ | Et₂O | 83 |
| 51 | Ph | H | H | Sc(OTf)₃ | THF | — |
| 52 | Ph | H | H | Sc(OTf)₃ | CH₂Cl₂ | 79 |
| 53 | Ph | H | H | Sc(OTf)₃ | CHCl₃ | 62 |
| 54 | Ph | H | H | Sc(OTf)₃ | MeNO₂ | 72 |
| 55 | Ph | H | H | Sc(OTf)₃ | EtCN | 8 |
| 56 | Ph | H | H | Sc(OTf)₃ | DME | 43 |

Preparation of (−)-2,3-Dichloro-4-(benzoylmethyl)-buten-2-olide (Scheme III, Formula 13, R¹=Ph, R²═R³═H)

A mixture of (R)-(+) or (S)-(−)-BINOL (29 mg, 0.10 mmol) and 4 Å molecular mg) in CH₂Cl₂ (5 mL) was stirred under a N₂ atmosphere. A solution of (i-PrOH)₂TiCl₂ (0.33 mL; 0.3 M in toluene) was added to the BINOL mixture and was stirred for 1 h. The insoluble material was removed via filtration and the filtrate was cooled at 0° C. Trimethyl-(1-phenyl-vinyloxy)-silane (Formula 14, R¹=Ph, R²═R³═H, 0.41 mL, 2.0 mmol) was added followed by portion-wise addition of mucochloric acid (Formula 7, 169 mg, 1.0 mmol) over 20 min. The mixture was stirred at 0° C. for 1.5 h and then allowed to warm to RT. After 9 d the reaction mixture was partitioned with H₂O (5 mL), the phases were separated, and the aqueous phase was extracted with CH₂Cl₂ (2×5 mL). The organic phases were combined, dried over MgSO₄, and concentrated to provide crude (−)-2,3-dichloro-4-(benzoylmethyl)-buten-2-olide as a dark orange oil; chiral HPLC analysis showed this material to be present with 81.0% ee.

The crude material was purified via chromatography to remove unreacted mucochloric acid, acetophenone, and most of the BINOL to provide the above-titled compound as a white powder. HPLC analysis: 98.6% chemical purity; 82.9% ee; [α]_{D}²⁰=−22.85° (c=16.8, MeOH) from (R)-(+)-BINOL. Trituration in Et₂O and removal of the solid (9.4% ee) followed by concentration of the filtrate, improved the chiral purity of the above-titled compound to 93.9% ee.

It should be noted that, as used in this specification and the appended claims, singular articles such as “a,” “an,” and “the,” may refer to a single object or to a plurality of objects unless the context clearly indicates otherwise. Thus, for example, reference to a composition containing “a compound” may include a single compound or two or more compounds. It is also to be understood that the above description is intended to be illustrative and not restrictive. Many embodiments will be apparent to those of skill in the art upon reading the above description. Therefore, the scope of the invention should be determined with references to the appended claims and includes the full scope of equivalents to which such claims are entitled. The disclosures of all articles and references, including patents, patent applications and publications, are herein incorporated by reference in their entirety and for all purposes.

What is claimed is:

1. A method of making a compound of Formula 2, 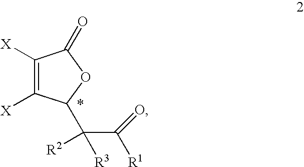
in which
X is halogen;
R¹ is C₁₋₆ alkyl, C₃₋₈ cycloalkyl, C₁₋₆ alkoxy, C₁₋₆ alkylthio, aryl, aryl-C₁₋₆ alkyl, aryl-C₁₋₆ alkoxy, or aryl-C₁₋₆ alkylthio; and
R² and R³ are independently hydrogen atom or C₁₋₆ alkyl, the method comprising reacting a compound of Formula 1, 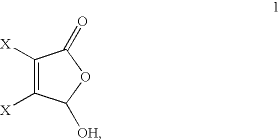
with a compound of Formula 3, 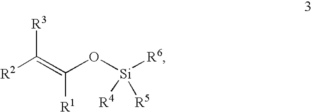
in the presence of a Lewis acid to yield the compound of Formula 2, wherein X in Formula 1 and R¹, R², and R³ in Formula 3 are as defined in Formula 2, and R⁴, R⁵, and R⁶ in Formula 3 are independently C₁₋₆ alkyl.

2. The method of claim 1, wherein the Lewis acid is chiral.

3. The method of claim 2, wherein the compound of Formula 2 is enantiomerically enriched.

4. The method of claim 1, wherein R¹ is C₁₋₆ alkyl, C₁₋₆ alkoxy, C₁₋₆ alkylthio, aryl, or aryl-C₁₋₆ alkyl.

5. The method of claim 1, wherein R¹ is methyl, methoxy, t-butylthio, or phenyl.

6. The method of claim 1, wherein R² and R³ are independently hydrogen atom or methyl.

7. The method of claim 1, wherein R⁴, R⁵, and R⁶ are each methyl.

8. A method of making 2,3-dihalo-4-(2-oxo-furan-5-yl)-buten-2-olide, the method comprising reacting a mucohalic acid with (furan-2-yloxy)-trimethyl-silane in the presence of a Lewis acid and solvent.

9. A compound of Formula 2, 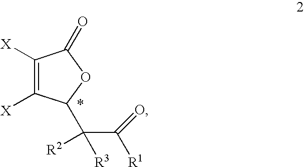
in which
X is halogen;
R¹ is C₁₋₆ alkyl, C₃₋₈ cycloalkyl, C₁₋₆ alkoxy, C₁₋₆ alkylthio, aryl, aryl-C₁₋₆ alkyl, aryl-C₁₋₆ alkoxy, or aryl-C₁₋₆ alkylthio; and
R² and R³ are independently hydrogen or C₁₋₆ alkyl.

10. The compound of claim 9, wherein the compound is enantiomerically enriched.

11. The compound of claim 9, wherein R¹ is C₁₋₆ alkyl, C₁₋₆ alkoxy, C₁₋₆ alkylthio, aryl, or aryl-C₁₋₆ alkyl.

12. The compound of claim 9, wherein R¹ is methyl, methoxy, t-butylthio, or phenyl.

13. The compound of claim 9, wherein R² and R³ are independently hydrogen atom or methyl.

14. The compound of claim 9 selected from:
2,3-dichloro-4-( 1-methyl-1-methoxycarbonyl-ethyl)-buten-2-olide;
(R)-2,3-dichloro-4-( 1-methyl-1-methoxycarbonyl-ethyl)-buten-2-olide;
2,3-dibromo-4-(1-methyl-1-methoxycarbonyl-ethyl)-buten-2-olide;
(R)-2,3-dibromo-4-(1-methyl-1-methoxycarbonyl-ethyl)-buten-2-olide;
2,3-dichloro-4-(t-butyl-thio-carbonylmethyl)-buten-2-olide;
(R)-2,3-dichloro-4-(t-butyl-thio-carbonylmethyl)-buten-2-olide;
2,3-dibromo-4-(t-butyl-thio-carbonylmethyl)-buten-2-olide;
(R)-2,3-dibromo-4-(t-butyl-thio-carbonylmethyl)-buten-2-olide;
2,3-dichloro-4-(benzoylmethyl)-buten-2-olide;
(R)-2,3-dichloro-4-(benzoylmethyl)-buten-2-olide;
2,3-dibromo-4-(benzoylmethyl)-buten-2-olide;
(R)-2,3-dibromo-4-(benzoylmethyl)-buten-2-olide; and
opposite enantiomers thereof.

15. A 2,3-dihalo-4-(2-oxo-furan-5-yl)-buten-2-olide.
